(19) United States
(12) Patent Application Publication
(10) Pub. No.: US 2024/0167201 A1
(43) Pub. Date: May 23, 2024

(54) CONDUCTIVE ELASTOMERIC FILAMENTS AND METHOD OF MAKING SAME

(71) Applicant: MYANT INC., Toronto (CA)

(72) Inventors: Ladan ESKANDARIAN, Toronto (CA); Milad ALIZADEH-MEGHRAZI, Toronto (CA); Amin MAHNAM, Toronto (CA); Tony CHAHINE, Toronto (CA); Sahar GOLMOHAMMADI ROSTAMI, Toronto (CA); Amirali TOOSSI, Toronto (CA)

(21) Appl. No.: 18/283,754

(22) PCT Filed: Oct. 26, 2021
(86) PCT No.: PCT/CA2021/051503

§ 371 Date: Sep. 22, 2023

Related U.S. Application Data
(60) Provisional application No. 63/164,183, filed on Mar. 22, 2021.

(51) Int. Cl. D02G 3/44; A61B 5/00; D01F 1/09; D01F 6/42; D01F 6/76; D02G 3/02; D02G 3/32; D03D 1/00; D03D 15/283; D03D 15/533; D03D 15/56; D04B 1/16; D04B 1/18; D04B 21/16; D04B 21/18

(52) U.S. Cl. CPC D02G 3/441; A61B 5/6804; D01F 1/09; D01F 6/42; D01F 6/76; D02G 3/02; D02G 3/32; D02G 3/448; D03D 1/0088; D03D 15/283; D03D 15/533; D03D 15/56; D04B 1/16; D04B 1/18; D04B 21/16; D04B 21/18; A61B 2562/125; D10B 2321/02; D10B 2321/121; D10B 2331/30; D10B 2401/063; D10B 2401/16; D10B 2509/00

(57) ABSTRACT

A biocompatible yarn comprising a conductive elastomeric filament, the conductive elastomeric filament comprising a elastomeric polymer and conductive filler.

### CROSS REFERENCE TO RELATED APPLICATION AND CLAIM OF PRIORITY

The present application claims priority to U.S. provisional patent application No. 63/164,183 filed on Mar. 22, 2021, the entire contents of which are hereby incorporated by reference.

### TECHNICAL FIELD

The disclosure relates generally to textile based electrodes, and more particularly to electrodes formed of conductive elastomeric material.

### BACKGROUND

Electrodes may be used for sensing biopotential signals or imparting electrical stimulation to a person's body. Wet gel has been used in electrodes to reduce impedance at the skin-electrode interface to improve sensing of biopotential signals or the ability to impart electrical energy to a person's body. However, application of a wet gel to a person's body may be difficult or undesirable for certain applications.

### SUMMARY

In one aspect, the disclosure describes a biocompatible yarn comprising: a conductive elastomeric filament, the conductive elastomeric filament comprising a elastomeric polymer and conductive filler.

In an embodiment, the conductive elastomeric filament has a ΔR/R₀ of less than 2.3 for 100% strain, where ΔR is change in resistivity (Ohm·m), and R₀ is resistivity at 0% strain.

In an embodiment, the conductive elastomeric filament has a Young's modulus in the range of 1-13 MPa.

In an embodiment, the biocompatible yarn comprises 39%-70% carbon and 30-61% elastomer. In an embodiment, the elastomer comprises silicone.

In an embodiment, the biocompatible yarn comprises at least one of carbon polyolefin (CPO); carbon styrene butadiene copolymer (CSBC); Carbon Silicone rubber (CSR1); and carbon silicone rubber (CSR2).

In an embodiment, the filament has a generally uniform diameter along a length of the filament.

In an embodiment, the filament is knitted and/or woven into the biocompatible yarn.

Embodiments may include combinations of the above features.

In another aspect, the disclosure describes a conductive elastomeric filament comprising a elastomeric polymer and conductive filler.

In an embodiment, the conductive elastomeric filament has a ΔR/R₀ of less than 2.3 for 100% strain, where ΔR is change in resistivity (Ohm·m), and R₀ is resistivity at 0% strain.

In an embodiment, the conductive elastomeric filament has a Young's modulus in the range of 1-13 MPa.

In an embodiment, the conductive elastomeric filament comprises 39%-70% carbon and 30-61% elastomer. In another embodiment, the elastomer comprises silicone.

In an embodiment, the conductive elastomeric filament comprises at least one of carbon polyolefin (CPO); carbon styrene butadiene copolymer (CSBC); Carbon Silicone rubber (CSR1); and carbon silicone rubber (CSR2).

In an embodiment, the filament has a generally uniform diameter along a length of the filament.

Embodiments may include combinations of the above features.

In another aspect, the disclosure describes a wearable dry textile comprising the conductive elastomeric filament and/or biocompatible yarn described in this disclosure.

In another aspect, the disclosure describes an electrode comprising the conductive elastomeric filament of the conductive elastomeric filaments described in this disclosure. The electrode configured for at least one of Electrocardiogram (ECG) measurement, electromyograms (EMG) measurement, electroencephalograms (EEG) measurement, Electrooculogram (EOG) measurement, Electrogastrogram (EGG) measurement, Functional Electrical Stimulation (FES), Transcranial Current Stimulation (TCS), High-Frequency Alternating Current Stimulation, Neuromuscular Electrical Stimulation (NMES), Transcutaneous Electrical Nerve Stimulation (TENS), Sensing pressure, Sensing strain, Heat generation, and/or creating a tactile sensation.

In an embodiment, the conductive elastomeric filament of the electrode is knitted and/or woven into a yarn, and the electrode is made from the yarn.

Embodiments may include combinations of the above features.

In another aspect, the disclosure describes a method of manufacturing a conductive elastomeric filament for an electrode. The method comprises providing elastomeric polymer pellets having desired material properties; combining the elastomeric polymer pellets and conductive filler together to form a conductive elastomer; extruding and drawing the conductive elastomer into a filament.

In an embodiment, the method comprises forming an electrode from the filament, the electrode configured for at least one of Electrocardiogram (ECG) measurement, electromyograms (EMG) measurement, electroencephalograms (EEG) measurement, Electrooculogram (EOG) measurement, Electrogastrogram (EGG) measurement, Functional Electrical Stimulation (FES), Transcranial Current Stimulation (TCS), High-Frequency Alternating Current Stimulation, Neuromuscular Electrical Stimulation (NMES), Transcutaneous Electrical Nerve Stimulation (TENS), Sensing pressure, Sensing strain, Heat generation, and/or creating a tactile sensation.

In an embodiment, the elastomeric polymer and conductive filler are comprise biocompatible material for forming a biocompatible yarn and/or filament.

In an embodiment, the elastomeric polymer is at least one of polyolefin, styrene butadiene copolymer, and silicone rubber; and the conductive filler is carbon black.

In an embodiment, extruding and drawing the conductive elastomer into the filament comprises melt spinning the filament. In another embodiment, the melt spinning temperature may be in a range of 130-360° C. In another embodiment, the melt spinning temperature is in a range of 250-310° C.

In an embodiment, the method comprises extruding and drawing the filament into a solvent bath. In another embodiment, the solvent bath is water.

In an embodiment, an elastomeric component of the elastomeric polymer comprises silicone.

In an embodiment, the filament has a generally uniform diameter along a length of the filament.

In an embodiment, the method comprises knitting and/or weaving the filament into a yarn.

Embodiments may include combinations of the above features.

Further details of these and other aspects of the subject matter of this application will be apparent from the detailed description included below and the drawings.

### DESCRIPTION OF THE DRAWINGS

Reference is now made to the accompanying drawings, in which:

FIG. 1 shows a skin-electrode interface for a wet electrodes;

FIG. 2 is an example electrode-skin interface for a dry electrode; and

FIG. 3 shows characterizations and electromechanical performance of four example conductive elastomeric filament (CEF) fibers in charts illustrating: (a) X-ray photoelectron spectroscopy (XPS) survey scans; (b) thermogravimetric analysis (TGA); (c) Strain-stress curves; (d) Strain-resistivity curves; and (e) Strain-relative resistance change (ΔR/R₀) curves.

FIG. 4 shows (a) illustrations of morphologies of CEF fibers; (b) illustrations of the design and structure of an example dry textile electrode; (c) illustrations of morphologies of CEF fibers dry textile electrodes.

FIG. 5 shows a chart of electrode-Skin impedance measurements performed on gel, CSBC, CPO, and CSR2 electrodes. Solid lines represent the mean, and shaded regions represent the standard deviation of the mean for each dataset.

FIG. 6 shows charts of on-skin ECG measurements using CEF fibers dry textile electrodes illustrating: (a) ECG recording methods and ECG trace features; (b) Electrocardiography (ECG) recordings performed using gel, CSBC, CPO, and CSR2 electrodes; (c) measurements of ECG R-peak amplitudes for the studied electrodes; (d) R-peak amplitude to peak-to-peak noise ratio for studied electrodes; (e) measurements of ECG T-peak amplitudes; and (f) T-peak amplitude to peak-to-peak noise ratio for studied electrodes.

FIG. 7 shows a chart of power spectral density curves of ECG recordings performed with (a) Gel, (b) CSBC, (c) CPO, and (d) CSR2 electrodes.

FIG. 8 shows a) illustrations of an example smart garment for ECG recording, specifically a knitted underwear garment with 5 embedded electrodes; b) charts of ECG traces recorded using CSR2 electrodes knitted in the band of an underwear garment. Recordings were done with unwashed and washed (30 cycles) garments when the subject was in three different positions: seated, supine position, and standing.

FIG. 9 shows charts illustrating the effect of washing on the performance of garment-embedded CSR2 electrodes when a subject was in seated position, specifically (a) measurements of ECG R-peak amplitudes before and after 30 wash cycles; (b) R-peak amplitude to peak-to-peak noise ratio before and after 30 wash cycles; (c) measurements of ECG T-peak amplitudes before and after 30 wash cycles; (d) shows T-peak amplitude to peak-to-peak noise ratio before and after 30 wash cycles where dashed lines represent the median of each dataset; and (e) power spectral density curves of 25 seconds long ECG recordings performed before and after 30 wash cycles.

FIG. 10 shows FTIR spectra of four CEF fibers according to this disclosure.

FIG. 11 shows charts illustrating (a) XPS high resolution scans of C1s and Si2p of four CEF fibers; and (b) Si2p spectra of four CEF fibers.

FIG. 12 shows Differential scanning calorimetry (DSC) of (a) cooling cycles, and (b) Heating cycles of CPO and CSBC CEF fibers.

FIG. 13 shows a knitted CEF fibers textile electrode (left, CPO electrode) and a failed one (right, CSR1 electrode).

FIG. 14 is a flow chart depicting a method of manufacturing an electrode.

FIG. 15 shows an example fabrication process of a dry textile electrode using melt-spun conductive elastomeric filament fibers.

### DETAILED DESCRIPTION

The following description relates to elastomeric materials for creating conductive elastomeric filament (CEF) fibers which may form textile-based electrodes suitable for e.g. sensing bioiopotential signals including Electromyogram (EMG), Electroencephalogram (EEG), Electrocardiogram (ECG), Electrooculogram (EOG), and Electrogastrogram (EGG), as well as applying current/voltage to body for Functional Electrical Stimulation (FES), Transcranial Current Stimulation (TCS), Neuromuscular Electrical Stimulation (NMES), Transcutaneous Electrical Nerve Stimulation (TENS), Sensing pressure, Sensing strain, Heat generation, High-Frequency Alternating Current Stimulation, and/or creating a tactile sensation. In some embodiments, the elastomer materials may comprise conductive thermoplastic elastomer materials. Example conductive elastomer materials according this disclosure may include at least one of Carbon Polyolefin (CPO); Carbon Styrene Butadiene copolymer (CSBC); Carbon Silicone rubber (CSR1); and Carbon Silicone rubber (CSR2). The composition of these material is shown below in Tables 1 and 2. Conductive elastomer materials according to this disclosure are not limited by thermoplastic elastomers of CPO, CSBC, CSR1 and CSR2; rather, any types of conductive elastomeric polymer may form a CEF according to this disclosure. In an embodiment, a dry-textile electrodes (e.g an Electrocardiogram (ECG) electrode) comprises conductive fibers of conductive elastomeric polymer(s), e.g. at least one of CPO; CSBC; CSR1; and CSR2. The description also describes method(s) of manufacturing the conductive elastomer materials, and CEF fiber, electrode(s), and textiles comprising the conductive elastomer materials disclosed herein. Example textile-based electrodes and method of manufacturing same are described International Application No. PCT/CA2020/051809 the entire disclosure of which is hereby incorporated by reference herein.

Although terms such as “maximize”, “minimize” and “optimize” may be used in the present disclosure, it should be understood that such term may be used to refer to improvements, tuning and refinements which may not be strictly limited to maximal, minimal or optimal.

The term “connected” or “coupled to” may include both direct coupling (in which two elements that are coupled to each other contact each other) and indirect coupling (in which at least one additional element is located between the two elements).

The term “substantially” as used herein may be applied to modify any quantitative representation which could permissibly vary without resulting in a change in the basic function to which it is related.

In modern society, health policies and patients' needs are continuously changing and individuals are becoming more and more conscious to the state of their health and wellbeing. Increases in the aging populations, the need for healthcare cost containment, and the need for improved methods of monitoring the quality of healthcare services' are some of the challenges modern societies currently face.^{[1,2]} The popularity of wearable devices has generated a great deal of clinical potential for continuous health monitoring of electrophysiological biomarkers.^{[3,4]} Electrophysiological signals targeted for wearable sensing, are electrical signals resulting from the electrochemical activity of the body's neural and muscular systems.^{[5]} Examples of such signals include electrocardiograms (ECG) from the heart muscle activity, electroencephalograms (EEG) from the brain activity, and electromyograms (EMG) from various muscles' activity.^{[6-8]} These biopotential signals may contain physiological data that can be used to diagnose, monitor, treat or manage various diseases.^{[9]} At present, devices capable of long-term measurement are not widely used as they can be expensive, bulky, conspicuous, and uncomfortable for users._{[10]}

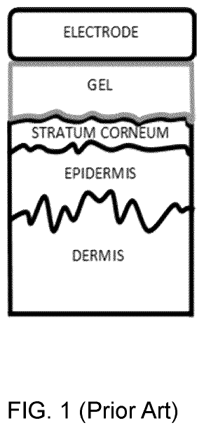

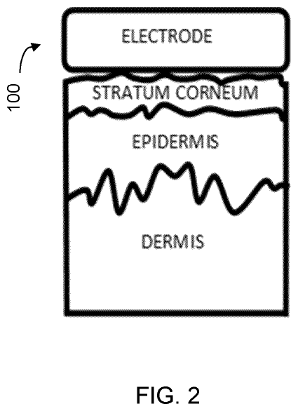

A necessary component of devices aiming to perform long-term electrophysiological monitoring are the sensors interfacing with the body, further referred to as biopotential electrodes. The existing gold-standard biopotential electrodes have a “wet” interface with the skin, employing an electrolytic gel as a conduit to transfer charge from the skin to the electrode.^{[10]} An example skin-electrode interface for a wet electrode is shown in FIG. 1. Ag/AgCL gel electrodes are made up of a skin-adherable material, to stabilize contact with skin. These electrodes are commonly used as disposable tools for biosignal recordings but may also be reused with a limited lifespan.^{[11]} Reusability challenge of gel electrodes is due to their wet and adhesive interface (i.e. electrolyte gel and adhesive) with the skin which over prolonged use can result in skin irritation and allergic reactions.^{[12]} Additionally, wet electrodes dry out overtime, causing degradation of signal quality; limiting their application for long-term or daily uses.^{[13]} These limitations have driven researchers to consider alternative dry electrodes for long-term biopotential measurement.^{[14,15]} Dry electrodes that are suitable for long-term biopotential measurement may need to meet five key design considerations. 1) Dry electrodes may have to be flexible to establish acceptable and consistent contact with the skin over various anatomical locations across the body.^{[12,16]} 2) Dry electrodes may have to be electrically conductive to allow for sensing of the body's electrophysiological activity.^{[17,18]} 3) Dry electrodes may have to be robust and durable in order to satisfy the requirements for long-term use.^{[19]} Specifically, they may need to withstand chemical, thermal and mechanical stresses involved in a user's day-to-day life. Examples include washing, abrasion, and stretch associated with wear conditions.^{[20]} This may be particularly important as many of the common materials used in electronics applications (e.g. metallic contacts) are sensitive to environmental conditions of daily living and can degrade when in contact with water or air.^{[10]} 4) As with any wearable device for long-term use dry electrodes may also need to be breathable to ensure skin health and user's comfort.^{[21,22]} Breathability is the ability of an electrode (which can be in a form of film, fabric, membrane, disc, etc.) to allow air and water vapour to pass through it.^{[23]} The lack of breathability could lead to lasting physiological and psychological effects by blocking airflow around the skin and causing irritation and inflammation.^{[24]} 5) Given that most continuous monitoring systems require multiple recording channels and therefore multiple electrode contacts spanning regions across various parts of the body, dry electrodes should allow for implementation of reasonable integration strategies.^{[25]} An example of a group of electrodes that are limited in this area, are those skin-printed/tattooed electrodes.^{[26-30]} An example skin-electrode interface for a dry electrode is depicted in FIG. 2.

Flexible conductive polymer dry contact electrodes have the potential to meet these requirements and overcome the disadvantages of wet gel electrodes and dry metallic electrodes (e.g. noncontact dry electrodes, micro/nano needle-based electrodes, rigid metal electrodes).^{[31-35]} The ability to seamlessly integrate electrodes in textiles may be extremely attractive and also promising for user adoption as part of the daily clothing industry as textiles. Smart garments are considered as the second closest surrounding in contact with the body after human skin. Smart garments create a bi-directional, and continuous medium between our bodies and the world around us, and offer many possibilities for monitoring diverse range of physiological parameters.^{[36]} Additionally, smart textiles are suitable for manufacturing, as they can be produced in a single-step by combining conductive and nonconductive materials via processes such as knitting.^{[19,37]}

Conductive fibers may be one of the smallest and one of the essential building blocks of wearable and flexible textile-electrodes. The limited availability of conductive elastomeric fibers that can be produced at a sufficiently large scale (>100 m) while meeting the mechanical properties and fiber size (diameter <1 mm) requirements has restricted their integration into smart textiles for practical electrophysiological sensing applications. Conductive fibers can be manufactured through various techniques such as fiber spinning, coating on fibers, and wrapping, twisting, and coiling other conductive materials such as stainless steel yarns or metallic wires with non-conductive fibers.^{[38-40]} Even though coating is a scalable and easy, problems may occur in the manufacturing process of conductive fibers, the poor adhesion and mechanical properties, mismatch between the fiber substrate, and the coating layer often result in the degradation of the sensing response, especially after knitting or numerous abrasive cycles such as daily wash and wear of consumer products.^{[41]} Conductive stretchable yarns produced using the wrapping, twisting, and coiling, are typically fabricated at small (centimeter) scale, which will not be sufficient for integration into textiles. Spinning conducting and elastomeric fibers may prevent the delamination of conducting fillers by integrating them within elastomeric polymers, such as silicone rubber, polyolefin, and polyurethane (PU).^{[42-44]} This approach is also scalable and has shown potential in producing conductive elastomeric filament (CEF) fibers at a length beyond a kilometer. However, imparting electrical properties while maintaining the stretchability in elastomeric polymer fibers has been a great challenge. This is because introducing conductive fillers often results in deterioration of spinnability (ability to form fibers) or can lead to fibers with low stretchability, which are not suitable for biosignal monitoring applications in a form of dry-textile electrodes since they don't have the necessary a sufficient mechanical properties to be knitted/woven into an electrode.^{[45-47]}

Conductive fibers may be used to form clothing that can sense bio potential signals or impart electrical stimulation to a person's body. Material development studies for conductive fibers have been undertaken to improve conductive fiber material compatibility with textiles and/or wearable electrodes. Durability, flexibility, breathability, being electrically conductive to allow for sensing of a body's electrophysiological activity, and the ability to impart electrical energy are among the characteristics of the conductive fibers studied according to this disclosure.

In an embodiment, this disclosure employs melt-spun conductive elastomeric filament (CEF) fibers as building blocks of dry-textile electrodes produced on industrial-scale 3D knitting machines. The properties of the conductive CEF fibers, including the electrical and mechanical properties, and the micro morphologies were investigated. The parameters of the preform, including the materials, the size and the shape of the fiber and their effect on knittability of dry-textile electrodes and electrodes' performance were systematically studied. In order to assess the performance of the dry textile electrodes in more realistic circumstances, underwear garments with embedded dry-textile electrodes were designed and knitted that can readily monitor ECG signals of a wearer in seated, standing and supine positions. To examine the durability of knitted dry electrodes, the effect of consumer wash and dry cycles on the performance of knitted dry textile electrodes was assessed by recording ECG signals using a 30 times washed garment with embedded electrodes. This work shows that a unique combination of electrical conductivity and stretchability of conductive elastomeric fibers enables them to be integrated into textiles for practical applications such as electrophysiological monitoring.

Aspects of various embodiments are described through reference to the drawings and examples.

### Examples

Four different types of conductive elastomeric filament (CEF) fibers were developed using elastomeric polymer matrix and conductive carbon black filler through melt spinning technique (See Table 1). The four example types of CEF fibers used in this study were sourced from Myant Inc., Ontario, Canada. Table 1 shows a list of fiber materials and their specifications. Diameter of each fiber was measured by ImageJ software using cross-sectional SEM images.

TABLE 1
Fiber Materials and specifications

| Fiber Code^{a)} | Polymer matrix | Diameter [μm] |
|---|---|---|
| CPO | Polyolefin (PO) | 765 |
| CSBC | Styrene Butadiene copolymer (SBC) | 596 |
| CSR1 | Silicone rubber (SR) | 592 |
| CSR2 | Silicone rubber (SR) | 462 |

^{a)}“C” as the first letter of each fiber code represents the conductive carbon filler

Among various types of thermoplastic elastomeric materials, polyolefin (PO) and styrene butadiene copolymer (SBC) thermoplastic elastomers (TPEs) were chosen as polymer matrices in this example due to their high strain ability at room temperature and thermoplastic behavior at elevated temperatures.^{[48]} This feature may make these materials very unique since they can be reprocessed and recycled easily. Phase separated systems with soft and rigid segments are the main components of TPEs. While the hard phase is responsible for the strength of the polymer, the soft phase allows elastomeric behavior at room temperature. Polyolefin blends TPEs (TPos) are primarily based on ethylene-propylene random copolymer (EPM) and isotactic polypropylene (iPP).^{[49]} TPOs are true thermoplastic elastomers, since neither of their hard and soft phases are cross-linked and both can flow. TPOs may be significant commercially due to their low cost, resilience to oil, solvents, and elevated temperatures, and high flexibility at low temperatures. SBC co-polymers are based on styrene and butadiene phases.^{[49]} While the styrene microphases are hard thermoplastic phases, the butadiene is the soft elastomeric phase.

Another group of polymers used in this disclosure is silicone rubber (SR) which is an elastomer material with a wide range of temperature resistance (−70° C. to 300° C.), good weatherability and moisture resistance, excellent oil and chemical resistance at high temperature and good mechanical properties.^{[48,49]} This material may be a very biocompatible elastomer, as it is commonly used in medical application and this feature makes it a strong candidate for textile-based biopotential electrodes.

Among different types of conductive fillers (CNTs, Graphene, Ag nanoparticles, etc), the carbon-based allotropes are usually preferred due to their low cost, low density, and superior chemical interaction with the base polymer materials. Application of safe and non-toxic materials in development of biopotential electrodes with direct skin contact is critical. Carbon Black (CB) particles are not cytotoxic and they are not soluble in either water, organic solvents, or biological fluids; therefore, CB would not be expected to be absorbed through the skin (US EPA 2005).^{[50-52]} Therefore, in these examples, carbon black (CB) fillers were chosen as conductive fillers in CEF fibers.

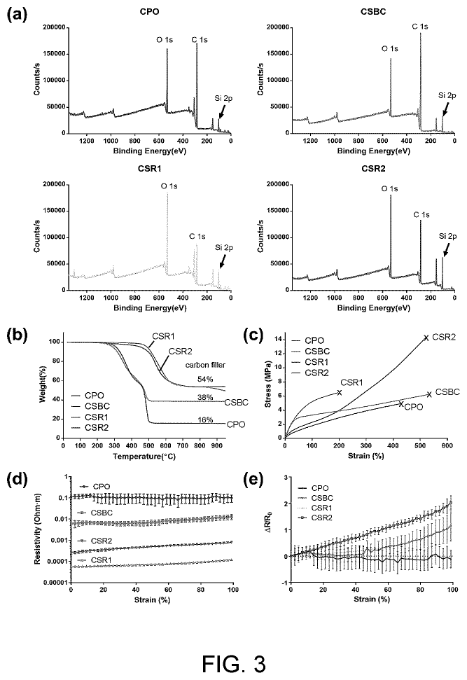

FIG. 3 illustrates characterizations and electromechanical performance of CEF fibers in charts: (a) XPS survey scans; (b) TGA; (c) Strain-stress curves—the cross symbols mean the yarn breaking point; (d) Strain-resistivity curves; (e) Strain-relative resistance change (ΔR/R₀) curves. The slope of the curve indicates the gauge factor. In (d) and (e), each yarn had 5 three segments measured, 15 cm each; the error bars (standard deviations) are not displayed if their heights are shorter than the symbols.

In terms of chemical compositions of four CEF fibers, XPS survey scans (See FIG. 3—charts (a)) showed that C, and Si dominated in all the CEF fibers. However, the Si contents were significantly higher in two silicone-rubber-based CEF fibers, namely CSR1 and CSR2 (See Table 2).

TABLE 2
XPS Relative Elemental Atomic Concentrations for CEF fibers.

| Yarn | % C | % O | % Si |
|---|---|---|---|
| CPO | 65 | 23 | 12 |
| CSBC | 70 | 18 | 12 |
| CSR1 | 39 | 37 | 24 |
| CSR2 | 51 | 24 | 25 |

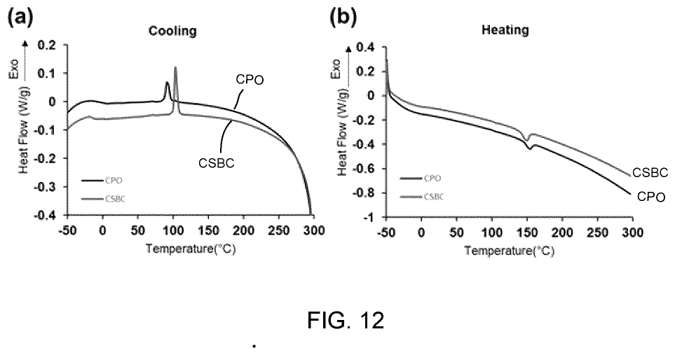

Higher silicone rubber content in CSR1 and CSR2 may explain why crystallization temperature (T_{C}) and melting temperature (Tₘ) were not observed in a narrow Differential scanning calorimetry (DSC) temperature range (−50° C. to 300° C.) (See Table 3 and FIG. 12 which illustrates DSC of (a) cooling cycles, and (b) Heating cycles of CPO and CSBC CEF fibers).

TABLE 3
Thermal Analysis of CEF fibers.

| | TGA | | | DSC | | | |
|---|---|---|---|---|---|---|---|
| Yarn | Residue wt % | T(onset) [° C.] | T(max) [° C.] | Tₘ[° C.] | ΔHₘ [J/g] | T_{c}[° C.] | ΔH_{c} (J/g) |
| CPO | 16 | 268 | 488 | 154 | 1.934 | 91 | 2.499 |
| CSBC | 38 | 225 | 483 | 150 | 3.324 | 104 | 4.832 |
| CSR1 | 54 | 427 | 557 | —^{a)} | — | — | — |
| CSR2 | 54 | 393 | 543 | — | — | — | — |

^{a)}Tₘ and T_{c} were not detected within the available temperature range of the DSC equipment(Q2000, TA Instruments; −50° C. to 300° C.).

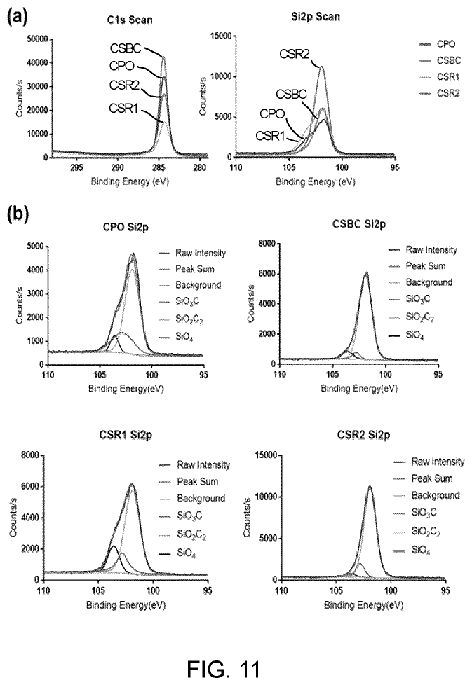

Silicone rubbers (with or without carbon fillers) usually have Tc below −50° C.; at the same time, Tₘ largely varies and sometimes could not be observed if co-components are well distributed, and no individual domain exists.^{[53,54]} High-resolution scans on C 1s and Si 2p (See FIG. 11—chart (a) illustrating XPS high resolution scans of C1s and Si2p, for four CEF fibers.) further revealed the binding energy of C (˜248 eV) and Si (˜102 eV), indicating the predominance of C-C (resulted from polymer backbones and carbon fillers) and organic Si in the CEF fibers. Introductions of organic Si (usually as co-blend siloxane polymers) into non-silicone-rubber-based CEF fibers such as polyolefin-based ones (POEs) and styrene-butadiene-based ones may improve their stretchability, tensile strength, and creep resistance.^{[55,56]} Peak deconvolutions on Si 2p scans (See FIG. 11—charts (b), illustrating the Si2p spectra of four CEF fibers) were based on three most common chemical environments in siloxane: SiO2C2, SiO3C and SiO4 with a binding energy of 101.9 eV, 102.8 eV and 103.6 eV, respectively; a higher presence of SiO3C and SiO4 means a higher crossing-linking and oxidation percentage in silicone rubbers.^{[57]} Thus, CSBC and CSR2 showed the least degree of cross-linked and oxidized siloxane among the four CEF fibers.

The resistances of CEF fibers mainly depended on the weight percentage of conductive carbon fillers inside the polymer blends, where a higher filler content resulted in a lower resistance. Carbon allotrope filler particles start to degrade at temperatures over 600° C., while elastomer materials including PO, SBC, and SR degrade at temperatures below 500° C.^{[48,58]} Therefore, performing TGA on CEF fibers up to 1000° C. will eliminate almost all the base polymer and leave a residual mass corresponding to the filler content in the samples. The residual weight percentage are presented in plotted TGA curves (FIG. 3—chart (b)) for the four CEF fibers). The residual mass values, in ascending order were 16%, 38%, 54% and 54% for CPO, CSBC, CSR1 and CSR2 fibers, respectively. The CSR1 fiber sample had an unusual TGA curve at the end plateau where near 1000° C. there was a small bu sharp loss in wt %.

Previous literature studies reported that inferior mechanical properties of conductive elastomeric composites is due to weak interfacial interactions between the conductive fillers and the polymer matrix.^{[59,60]} In order to successfully develop knitted electrode for electrophysiological applications, the mechanical properties of CEF fibers should also be paid attention. DMA results (FIG. 3, chart (c) and Table 4) showed that Young's modulus and strain-stress behaviors of four CEF fibers all fell into the range of typical elastomers, which usually have 1-10 MPa for Young's modulus and relatively flat curves.^{[61]} However, CSBC fiber shared certain similarities with semi-crystalline polymers in terms of the strain-stress curve shape, which suggested CSBC possessed slight crystallinity. CSR1 was the only CEF fiber that broke below 207% elongation; as discussed in following sections of this research study, knitted electrodes of each CEF fiber were fabricated except CSR1 fiber which was due to its breakage issues related to its lower strainability under tension.

TABLE 4
Tensile Properties of CEF fibers

| | Max | Tensile | Young's |
|---|---|---|---|
| Yarn | Strain[%] | Strength[MPa] | Modulus[MPa] |
| CPO | 419.42 ± 70.84 | 4.69 ± 0.36 | 3.38 ± 0.43 |
| CSBC | 523.36 ± 10.04 | 5.97 ± 0.39 | 9.63 ± 1.91 |
| CSR1 | 207.08 ± 44.59 | 6.38 ± 0.31 | 11.95 ± 0.80 |
| CSR2 | 494.78 ± 27.34 | 11.67 ± 3.28 | 4.96 ± 1.19 |

Each yarn had five segments measured

Apart from the interfacial interactions between the conductive fillers and the polymer matrix, the filler content influences both the electrical and mechanical performance of CEF fibers significantly. Therefore, the electromechanical properties of CEF fibers were characterized by elongating the CEF fiber upon 100% strain at ˜100 mm/min (FIG. 3, charts (d) (e)). In contrast to conductive filaments for strain-sensor applications, in order to create a homogenous textile electrode the resistance sensitivity of CEF fibers to strain needs to be low. Specifically, the responses can be linear (CSR2), divided into insensitive and linear regions (CSBC and CSR1), or insensitive (CPO). The different conductive networks within the polymeric matrix may lead to these various behaviors. If the percolated channels were stretched but the interconnected conductive networks did not cleavage, the resistance change with strain was ignorable, which applied to CPO, or CSBC and CSR1 with <50% strain; when the conductive networks experienced cleavages and reconstructions, a relatively low resistance increase to strain would be observed, which applied to CSR2, or CSBC and CSR1 with 50-100% strain.^{[62]} The insensitive resistance change under elongation observed here was crucial to utilize these CEF fibers as textile electrodes for chronic electrophysiological monitoring applications. As the strain in a shirt can be up to 20% around shoulders,^{[63]} the dry textile electrodes embedded in the smart garment could face various stretching in daily usages. All the four CEF fibers in this study did not exhibit dramatic resistance raises (all within 100%) with 0-50% strain and remained highly conductive, ensuring the relative stable resistance of dry textile electrodes when wearing.

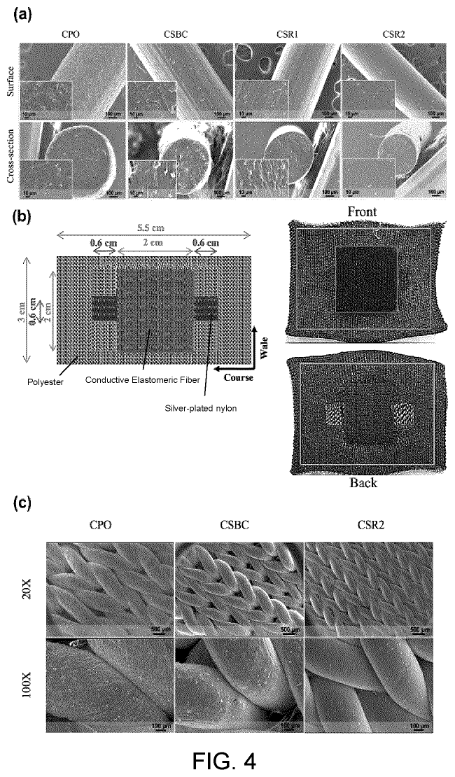

FIG. 4, illustration (a), shows Morphologies of CEF fibers characterized by SEM. The simulated fabric view of the electrode prior to knitting. The fabric structure and loop formation are chosen based on the yarn parameters. CEF fibers dry textile electrodes. FIG. 4, illustration (b) shows the design and structure of dry textile electrodes. FIGS. 4, illustration (c) shows Morphologies of CEF fibers dry textile electrodes characterized by SEM.

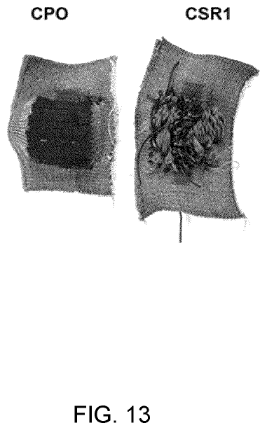

Morphological investigation of the CEF fibers and knitted dry textile electrodes using scanning electron microscopy (SEM) revealed the structure of fibers before and after knitting process. As shown in FIG. 4, chart (a), no obvious fractures and defects were detected in surface or cross-sectional images of CEF fibers, showing the melt spinning process was suitable for continuously manufacturing conductive fibers. All four CEF fibers possessed homogenous round cross-sections. CSR2 fiber had the smallest diameter of 462 μm, followed by CSR1, CSBC and CPO with the diameter of 592 μm, 598 μm and 765 μm, respectively. As shown in FIG. 4, chart (b), an example 3D electrode has a double-knit structure with a layer of conductive elastomeric fibers knitted on the front layer which is the layer that will be in contact with the skin when the electrode is placed on the body. By using the tuck stitch operation, the passive yarns that are knitting the technical back of the double-knit structure, can tuck and float behind the CEF fiber to create a 3D pattern. Tuck stiches can form holes on the surface of the fabric which can increases the air permeability of the knit fabric by allowing more air to pass through the surface of the electrode.^{[64]} Applying this knitting technique may lead to a structure with a higher brethability for biopotential electrodes. Silver plated nylon yarn is knitted at the technical back of the fabric, creating two small squares on both sides of the electrode to provide conductive termination points for integration. All the CEF fibers, except CSR1, were successfully knitted into dry textile electrodes following the pattern shown in FIG. 4, chart (b). CSR1 was unable to be manufactured into proper electrodes (see FIG. 13—A comparison between a successfully knitted CEF fibers textile electrode (left, CPO electrode) and a failed one (right, CSR1 electrode)) because the filament failed to endure the forces and stresses applied by the knitting machine. This may be due to CSR1 being the most rigid filament (the highest Young's modulus) with the lowest maximum strain (Table 4). SEM images of dry textile electrodes made from CPO, CSBC and CSR2 (FIG. 4, chart (c)) showed that the yarns remained intact after knitting, and the surface morphologies remained similar to the unknitted yarns (FIG. 4, chart (a)). This indicated the CEF fibers were very flexible and could be turned into various shapes without being damaged.

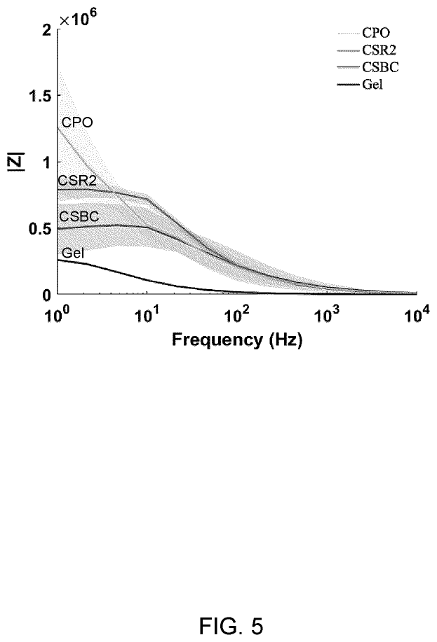

To assess the electrical properties of various CEF fibers described in Table 4, swatch electrodes were knitted as shown in FIG. 4, chart (b). Impedance measurements were done on knitted textile electrodes as well as gel adhesive electrodes as a baseline for electrophysiological recordings. Impedance measurement results are illustrated in FIG. 5, showing the gel electrodes to have the lowest impedances, followed by CSBC electrodes. Specifically, FIG. 5 illustrates electrode-skin impedance measurements performed on gel, CSBC, CPO, and CSR2 electrodes (n=3/electrode material). Solid lines represent the mean, and shaded regions represent the standard deviation of the mean for each dataset. There is a direct correlation between the skin-electrode impedance and strain-relative resistance change (ΔR/R₀) of CEF fibers shown is FIG. 3, chart (e). As can be seen in FIG. 4, chart (c), the CEF fibers are elongated during knitting to create a loop. As shown in FIG. 3, chart (e), the relative resistance change of CSR2 fiber is more susceptible to change under strain than the CSBC fiber. Therefore, even though the conductivity of the CSR2 fiber was initially higher than the CSBC fiber, the skin-electrode impedance of knitted CSR2 electrodes was higher compared with electrodes made of CSBC fibers.

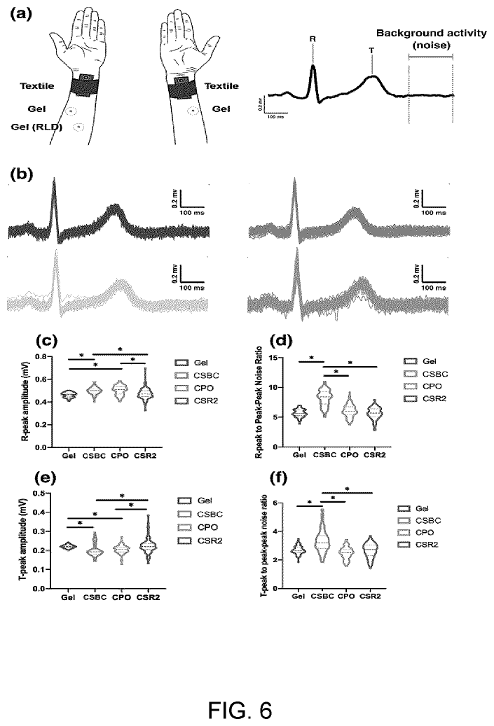

FIG. 6 illustrates on-skin ECG measurements using CEF fibers dry textile electrodes. In chart (a) of FIG. 6, ECG recording methods and ECG trace features. Chart (b) of FIG. 6 shows Electrocardiography (ECG) recordings performed using gel, CSBC, CPO, and CSR2 electrodes. Chart (b) of FIG. 6 shows one hundred overlaying P-QRS-T complexes recorded consecutively using these electrodes. Chart (c) of FIG. 6 shows measurements of ECG R-peak amplitudes for studied electrodes (n=3 pairs of electrodes×100 ECG pulses/electrode pair). Chart (d) of FIG. 6 shows R-peak amplitude to peak-to-peak noise ratio for studied electrodes. Chart (e) of FIG. 6 shows measurements of ECG T-peak amplitudes for studied electrodes (n=3 pairs of electrodes×100 ECG pulses/electrode pair). Chart (f) of FIG. 6 shows T-peak amplitude to peak-to-peak noise ratio for studied electrodes.

Electrocardiography was chosen for on-body testing of electrodes due to its wide use in electrophysiological monitoring applications. ECG electrodes were placed over the wrists and forearms as shown in FIG. 6, chart (a). Measured ECG recordings are shown in FIG. 6, chart (b). As shown in charts (c) and (e) of FIG. 6, R-peak amplitudes were largest in recordings performed with CSBC and CPO electrodes (p<0.0001 for Gel vs. CSBC, Gel vs. CPO, CSBC vs. CSR2, and CPO vs. CSR2), while T-peaks were largest in CSR2 and gel adhesive electrodes (p<0.0001 for Gel vs. CSBC, CSBC vs. CSR2, and CPO vs. CSR2; and p=0.0013 for Gel vs. CPO). Importantly, as a measure of signal to noise ratio (SNR), the ratio of R-, and T-peaks to peak-to-peak background noise, were largest in CSBC electrodes (p<0.0001 for all comparisons). No significant difference was observed between the SNR of CSR2 and CPO electrodes relative to gel adhesive electrodes (FIG. 6, charts (d) and (f)).

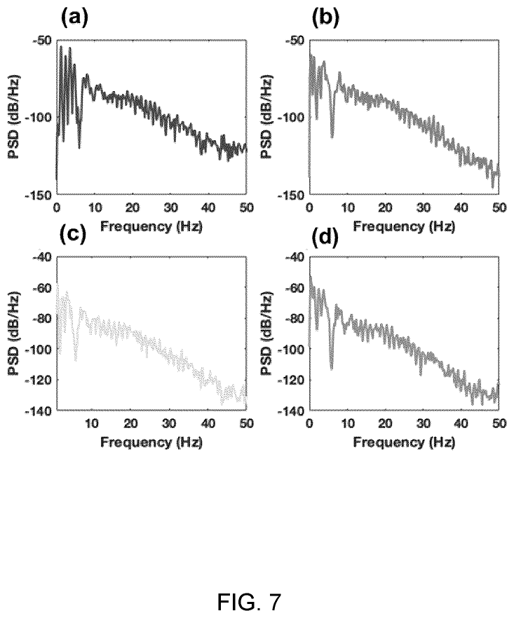

FIG. 7 illustrates power spectral density curves of ECG recordings performed with (a) Gel, (b) CSBC, (c) CPO, and (d) CSR2 electrodes. A comparison between the frequency distributions of recorded ECGs using various electrodes was done using PSD calculations (See FIG. 7). PSD curves of textile electrodes were found to have a high correlation with that of the gel adhesive electrode. R-squared values were 0.80, 0.80, 0.73 for CSBC, CPO, and CSR2 electrodes relative to gel electrodes (p<0.0001 for all pairs), respectively (See Table 5).

TABLE 5
R-squared values for correlation calculations
of Gel vs. CSBC, CPO, and CSR2 electrodes.

| | Gel vs. | Gel vs. | Gel vs. |
|---|---|---|---|
| Correlation | CSBC | CPO | CSR2 |
| Pearson r | | | |
| r | 0.8955 | 0.8962 | 0.8532 |
| 95% | 0.8746 to 0.9132 | 0.8754 to 0.9138 | 0.8245 to 0.8775 |
| confidence | | | |
| interval | | | |
| R squared | 0.8020 | 0.8032 | 0.7279 |
| P value | | | |
| P (two-tailed) | <0.0001 | <0.0001 | <0.0001 |
| P value | **** | **** | **** |
| summary | | | |
| Significant? | Yes | Yes | Yes |
| (alpha = 0.05) | | | |
| Number of | 411 | 411 | 411 |
| XY Pairs | | | |

Collectively, the result shown in Table 5 suggest that dry textile electrodes described in this disclosure allow for high-fidelity ECG recordings comparable (i.e. CSR2 and CPO) or superior (i.e. in the case of CSBC) to that of gel electrodes. ECG recordings performed with textile electrodes also have a similar frequency distribution as that of the gel electrodes.

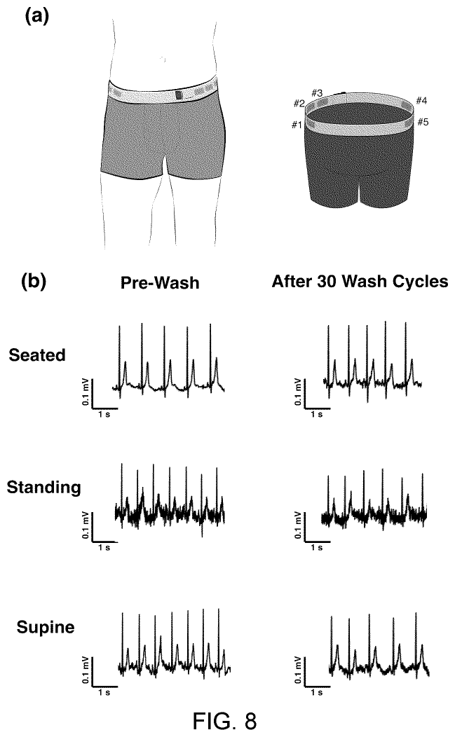

FIG. 8, chart (a) shows an example smart garment for ECG recording. Specifically, a knitted underwear garment with 5 embedded electrodes is illustrated. Contacts 1 and 5, 3 and 4, and 3 and 5, form corresponding recording channels. FIG. 8, chart (b), shows ECG traces were recorded using CSR2 electrodes knitted in the band of an underwear garment. In order to assess the performance of the dry textile electrodes in more realistic form factors and testing circumstances, underwear bands with embedded electrodes were designed and knitted (see FIG. 8, illustration (a)). ECGs were measured from three pairs of electrodes in three differential recording channels, simultaneously. Recordings were done with unwashed and washed (30 cycles) garments when the subject was in three different positions: seated, supine position, and standing. Specifically, recordings were done as an individual wearing the garment was in seated, standing, and lying down (supine) positions. Results are shown in FIG. 8, charts (b) showing the robustness of high-fidelity ECG recordings with embedded CSR2 electrodes. The average R-peak and T-peak to noise peak-to-peak noise ratio were 11.59 and 4.5, respectively, in seated position. A comparison between FIG. 6 (charts (c)-(f)) and FIG. 8 reveals that the absolute values of the R and T peaks are smaller in the measurements done with the garment relative with those done with pairs of surface electrodes. This difference is likely due the fact that the recordings from the garment are performed from the waist, while the recordings with individual electrode pairs are done over the forearms.

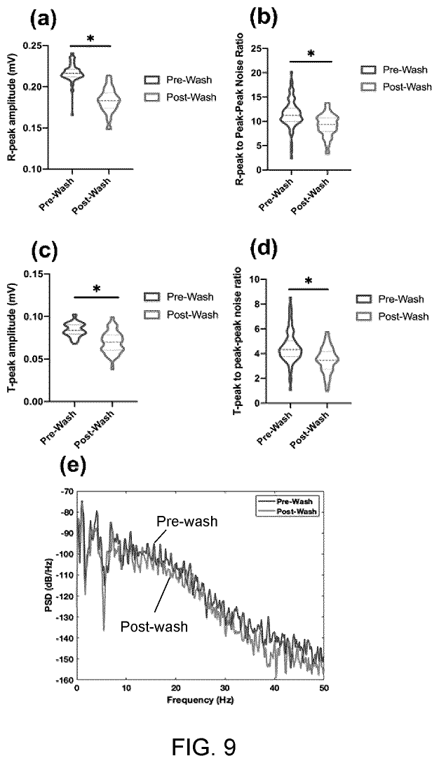

FIG. 9, charts (a)-(e) illustrate the effect of washing on the performance of garment-embedded CSR2 electrodes. Recordings were done while the subject was in seated position. In order to assess the effect of consumer wash and dry cycles on the performance of smart textile with embedded CEF fibers electrodes, a smart garment with five knitted electrodes made out of CSR2 yarns was washed 30 times according to the American Association of Textile Chemists and Colourists (AATCC) home laundry washing test method using a commercial washing machine with detergent. More details related to the employed washing method is provided in the methods section. More specifically, FIG. 9, chart (a), shows measurements of ECG R-peak amplitudes before and after 30 wash cycles (n=3 pairs of electrodes×85 ECG pulses/electrode pair); chart (b), shows R-peak amplitude to peak-to-peak noise ratio before and after 30 wash cycles; chart (c) shows measurements of ECG T-peak amplitudes before and after 30 wash cycles (n=3 pairs of electrodes×85 ECG pulses/electrode pair); chart (d) shows T-peak amplitude to peak-to-peak noise ratio before and after 30 wash cycles. dashed lines represent the median of each dataset; and chart (e) shows power spectral density curves of 25 seconds long ECG recordings performed before and after 30 wash cycles.

Before and after completing the 30 wash cycles, ECG recordings were performed consistent with the methods described in this disclosure with the subject seated. Results are shown in FIG. 8 (chart (b)) and FIG. 9 (charts (a)-(e)). As shown in these figures, high fidelity recordings can still be obtained with the electrodes even after 30 wash cycles. Nevertheless, as shown in FIG. 9 (charts (a)-(e)), a small, but statistically significant (p<0.0001 for all comparisons) reduction in the signal to noise ratio of the R- and T-peaks is observed after the wash cycles. The average R- and T-peak to peak-to-peak noise ratio reduced from 11.59 to 9.205, and from 4.517 to 3.499, respectively. It is also important to note that the frequency distribution of the ECG signals remained similar to the pre-wash state, as evidenced by a high correlation (r-squared=0.93, p<0.0001) between the PSDs of ECG recordings before and after this step (FIG. 9, chart (e)). Collectively, these results suggest that CSR2 yarns are a robust candidate for use in smart garments, especially those for electrophysiological monitoring applications.

CEF fibers and dry textile electrodes for ECG monitoring may be manufactured according to this disclosure. In an example, 3D shape textile electrodes may be knitted on a 7.2 gauge flat bed knitting machine (CMS ADF 32W E7.2). Textile electrodes may be programed and simulated using Stoll's M1PLUS® software. Surrounding fabric of the textile electrode may be polyester/spandex yarns (OMTEX/Invista). A underwear band, e.g. the underwear illustrated in FIG. 8, was knitted on an 18 gauge flat bed knitting machine (CMS ADF 530-32 BVV) with CSR2 fiber given this fiber's smaller diameter (which may be desirable for knitting considerations) and acceptable functional performance. By using the knitting machine capabilities, the five 3D-shaped electrodes (each 1.5×3.5 cm) were knitted on a compressive band as a single unit underwear garment in one piece. The targeted skin-electrode pressure was about 20 mmHg. Surrounding fabric of underwear textile electrodes comprised Nylon/spandex yarns (NOVAR/Invista). The same knitting operations for single electrodes may be applied for the example electrodes of this disclosure embedded in the underwear band structure. The consistency of the knitting parameters and principles enable the engineers to increase the number of the electrodes from one to five in the same pattern (see e.g. FIG. 8, chart (a)).

Electrical resistance and elongation measurement according to this disclosure were conducted as disclosed below. The resistance measurements were taken with a dual measurement multimeter (GW Instek GDM-8351 Dual Measurement Multimeter). The sampling rate of the multimeter was set to 0.1 seconds. A universal tensile tester was used to stretch a 7 cm segment of the CEF fiber of interest at a constant speed of 100 mm/min. The reliability of this resistance measurement protocol was evaluated by measuring the resistance of 5 samples of each CEF fiber.

Given that each CEF fiber had different diameters, resistivities were used instead of the resistances in the analysis and plotting (e.g. FIG. 3, chart (d)). The resistivity was calculated based on the Equation 1:

$$ρ = \frac{RA}{l} \vert \qquad Equation 1$$

Where ρ stands for resistivity (Ohm·m), R stands for resistance (Ohm), A stands for the cross-section area of the CEF fiber (m2), and I stands for the length of the CEF fiber (m).

Characterization of conductive elastomeric fibers and knitted electrodes according to this disclosure was conducted as discussed below. A scanning electron microscopy (JEOL, JSM 1000) was used to evaluate the morphological characteristics of the conductive elastomeric fibers and knitted electrodes. The filaments were gold sputtered for 1 min (thickness: 5-10 nm) before imaging; the textile electrodes were imaged without sputtering. Surface chemistry of conductive elastomeric fibers was analyzed by X-ray photoelectron spectroscopy (XPS) performed on a K-Alpha XPS apparatus (Thermo-Scientific) on an aluminum substrate with copper pin holders. A thermogravimetric analysis (TGA Q50, TA Instruments) was used to determine the conductive filler content of the conductive elastomeric fibers. Samples were cut to a weight of approximately 20 mg and tested to 1000° C. in a nitrogen atmosphere with a heating rate of 20° C./min.

A Dynamic Mechanical Analyzer (DMA Q800, TA Instruments) was used to perform the tensile stress-strain tests. The fibers were exposed to a ramped force (force ramp rate of 3 N/min) and the resultant deformation (strain) is monitored until the fiber's failure. All the experiments were done at room temperature. The reliability of this resistance measurement protocol was evaluated by measuring the resistance of 5 samples of each conductive elastomeric fiber. Five replicates of each CEF fiber were used in order to confirm the results presented in this work. The apparent tensile stress was determined using the cross-section of each fiber and the strain computed from the crosshead displacement. The apparent Young's modulus was computed from the first linear section of the stress-strain curve in the reloading phase.

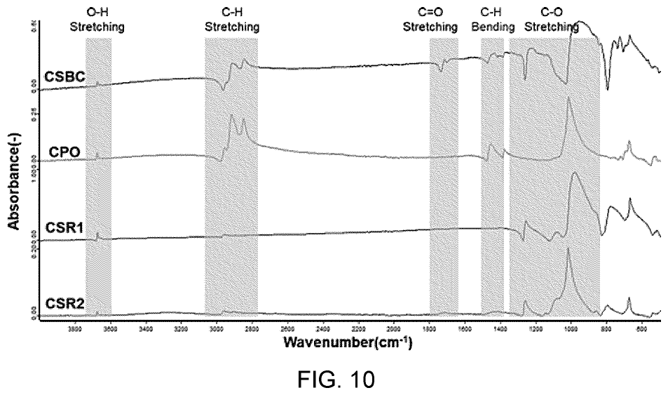

A Fourier transform infrared spectroscopy (FTIR, Alpha, Platinum-ATR, Bruker, Inc.) was conducted in the spectral range from 4000 to 400 cm-1. The spectra were the results of 64 interferograms at a spectral resolution of 4 cm-1. Results are shown in FIG. 10 (FTIR spectra of CSBC, CPO, SCR1, CSR2 fibers).

A Differential scanning calorimetry (DSC, Q2000, TA Instrument) was used to study the glass transition and melting behavior, During DSC testing, samples were heated from −50° C. to 300° C. at a heating rate of 10° C./min under dry nitrogen atmosphere.

Laundry Washing described in this disclosure was conducted as discussed below. Underwear garment prototype(s) with embedded electrodes were washed 30 times according to the American Association of Textile Chemists and Colourists (AATCC) home laundry washing test method using a commercial washing machine (Whirlpool WED5600X) under a normal laundry cycle for a small load with cold water using AATCC Standard Reference Detergent Without Optical Brightener (SDL Atlas, USA). Each sample prototype was placed in a mesh laundry bag during laundering. After each laundering cycle, the sample was laid flat and left to dry at room temperature prior to the next wash cycle.

Skin-electrode electrical impedance measurements according to this disclosure were performed using the measurement protocols described by Spach et al.^{[65]} Galvanostatic electrical impedance spectroscopy measurements were done over 1 to 10 KHz frequency range and 10 uA current range, using an Ivium Vertex One potentiostat (Ivium Technologies, Eindhoven, Netherlands). Swatches were knitted containing electrodes with each of the described CEF fibers materials (n=6/material type). Knitted swatch electrodes were 2 cm×2 cm and square in shape (see FIG. 4, chart (b)). Impedance measurements were also done on gel adhesive electrodes as the gold standard electrodes for electrophysiological recordings.

Electrocardiogram measurement according to this disclosure was tested in an example with pairs of surface electrodes. Swatch electrodes were knitted with each of the described CEF fibers (n=6/material type). In order to compare and validate the recording fidelity and performance of textile electrodes, simultaneous measurements were also done with gel adhesive electrodes. FIG. 6, illustration (a), shows the placement of textile and gel electrodes on the subject's forearms. Recordings were done when the subject was sitting, at rest. Textile electrodes were fixed onto the skin using adjustable straps around the forearm. The pressure between the dry textile electrodes and the skin (applied by the straps) was controlled by calibrated pressure measurements at the time of their placement. The targeted skin-electrode pressure was 20 mmHg. ECG recordings were done simultaneously from the gel and textile electrode pairs using an 8-channel OpenBCI Cyton biosensing system (OpenBCI company, Brooklyn, USA). Recordings were done at a 250 Hz sampling frequency. All analyses were performed using a custom written program in Matlab (Ver. R2020a, Mathworks company, Natick, USA). In order to remove baseline drift and low-frequency motion artifacts, the data was filtered using a second order butterworth high-pass filter with a corner frequency of 0.5 Hz. R- and T-peaks were measured and their ratio to average peak-to-peak background noise was calculated. Welch's estimated power spectral densities were also computed for recordings from each electrode type and their correlations were calculated using Pearson correlation coefficient. All statistical analyses were performed using the Graphpad Prism software (Ver 8, Graphpad Software Inc, San Diego, USA). Group comparisons were done using ANOVA tests, and Tukey's multiple comparisons tests.

Testing with surface electrodes embedded in a garment was done using garment-based ECG recordings simultaneously from textile electrode pairs shown in FIG. 8 using a Myant's Skiin Pod (Myant Inc., Toronto, Canada). Recordings were done at a 320 Hz sampling frequency. Same methods were used for electrophysiological data analysis as those described for tests with pairs of surface electrodes (part-a of this section). On body impedance and ECG measurements of dry textile electrodes as well as the underwear with embedded textile electrodes were only performed on one individual to minimize the impact of confounding variables that can affect signal quality or skin impedance between measurements and individuals such as skin hydration, motion, electrode placement, and sweating 56-60. Future studies will focus on evaluating the performance of these electrodes with a larger sample size.

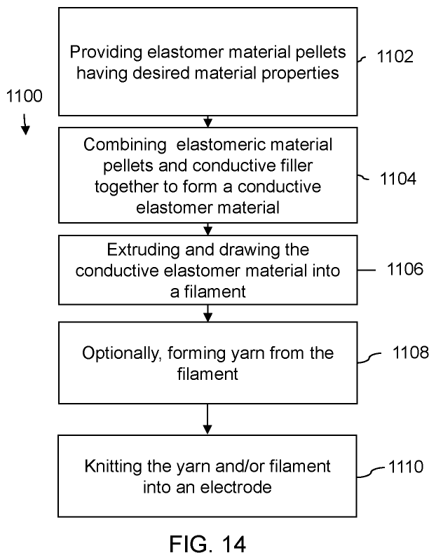

FIG. 14 is a flow chart depicting an example process 1100 for manufacturing an electrode. At block 1102, elastomeric material pellets, e.g. elasomeric polymer pellets, having desired elastomer material properties are provided.

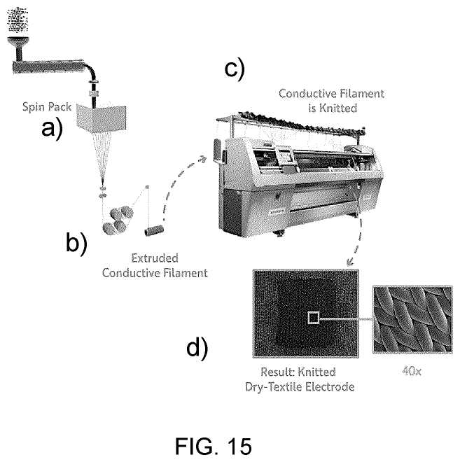

At block 1104, elastomer material pellets and conductive filler are combined together to form a conductive elastomeric material. In an example, the conductive elastomeric material pellets and conductive filler may be compounded to mix the conductive fillers with polymer forming conductive polymer pellets. In an embodiment, conductive filler may be carbon black particles. Carbon-based materials such as carbon nanotubes, graphene, carbon black, acetylene black, and mixture thereof may also be as conductive filler. Conductive filler is not limited to carbon material, and may be inorganic compounds such as MXenes and/or metallic nano-fillers such as silver, gold or brass. Conductive fillers may be selected based on (1) Biocompatibility of the conductive filler, (2) Size and morphology, (3) Surface area, (4) Percolation rate, (5) Conductivity, (6) Spinnability. The conductive polymer pellets may be added to a hopper of a melt spinning machine as shown in FIG. 15, illustration a). In an embodiment, the melt spinning machine can be mono-, bi-, tri-, quad-components. Depending on the desired properties of the filament and/or filament yarn, the composition of the yarn may be selected from different components. Each component may have a separate hopper/feeder and heating zone to melt each of the components together—including the conductive polymer pellets which is melted to form conductive elastomeric polymer component.

Extrusion temperature, melt intrinsic viscosity, filler content, feed rate, and take-up velocity are influential variables for conductive elastomeric fibers in melt spinning, as they affect the molecular orientation and crystallinity of as-spun/drawn fibers. Spinning temperature may affect the melt viscosity and thereby the flow distribution through the spinneret. Reduced melt viscosity variations in a spinpack from reduced intrinsic viscosity, residence time, and temperature gradients will yield reduced denier and orientation variations from filament-to-filament. In an embodiment, the conductive elastomeric polymer pellets and conductive filler may be melted at a temperature from 130 C to 360 C. In another embodiment, the conductive elastomeric polymer pellet and conductive filler may be melted together at a temperature below 130 C. In another embodiment, the conductive elastomeric polymer pellet and conductive filler may be melted together at a temperature from 250 C to 310 C. Components of the filament yarn may include conductive polymer (such as conductive TPE), self-healing materials, far infrared (FIR) particles and microcapsules of phase-change materials for thermal regulation.

At block 1106, the conductive elastomeric material, e.g. elastomeric polymer, may be extruded and drawn into a filament. As a filament is formed, the molecules of the filament are oriented simultaneously, e.g. in a spin column. Smaller diameter fibers (overall from filament-to-filament and along each filament) may each have higher as-spun orientation (birefringence) than the larger ones. In a typical fiber process, a multifilament as-spun yarn may be drawn (extended) 3-5 times its original length to orient the molecules further and achieve its final desired tensile properties (e.g. tenacity, % elongation, modulus, etc.). Providing increased uniformity of as-spun fiber properties, particularly denier and attendant orientation along the filament, influence the improvements in properties, throughputs, and quality of drawn fibers. Uniformity of the fiber quality, in particular its tensile properties, may be influenced by the uniform structure (diameter) of the continuous fiber. Having thin and thick places along the filament would create non-uniformity which will affect the mechanical properties and ultimately the ability of the fibers to form a textile, e.g. by knitting, weaving, and/or embroidering.

Continuing the above example, as FIG. 15 illustration a), the conductive elastomeric polymer may be extruded and drawn in the melt spinning machine to form filament(s), which may be combined with filaments of other components at a spinneret (shown in FIG. 15 illustration a) to form a filament yarn as discussed at block 1108, which may then be directly solidified by cooling. In an example, spinneret throughput may be 0.3 to 4 g/min. Different filament structures may be created, such as monocomponent with different diameters (e.g. 50 microns to 400 microns) and bicomponent structures (e.g. core-sheath, lobal, side-by-side, segmented, and Islands-in-the-sea). Filaments may then be wound and collected on a take-up wheel illustrated in FIG. 15 illustration b). Filament from the spinneret may be collected at various speeds. The take-up speed, i.e. the rate at which the filament is pulled from the spinneret, may stretch the filament to a desired diameter or cross-sectional area. In an embodiment, filament from the spinneret may free fall to a winding apparatus. In another embodiment, take-up speed may range from 20-30 rpm

In an example, the spinneret can be configured to provide a different cross sectional shapes and diameters of extruded filaments. In an embodiment, diameter of the spinneret can be between 50 micron to 1 mm. The extruded filament may be drawn to improve the crystallinity and create thinner filaments. Drawing a filament may increase the molecular orientation and strength of the fibers while decreasing their extensibility compared to as-spun fibers. During an example drawing process, as-spun filaments may be stretched up to 5 times of their original length. This drawing process may happen at a temperature from 10° C.-100° C. above the glass transition temperature (Tg) of the polymer, and subsequently the filaments may be heatset to impart dimensional stability. Spinning temperature and drawing steps may affect the orientation of the polymer molecules which subsequently affects the tensile properties of the final fiber. In an embodiment, the diameter of the extruded filament may be drawn to have a diameter in the range of 100 to 500 micron. In an example, the properties (e.g. spinnability, biocompatibility, and conductivity) of a monocomponent filament structure may solely depend on the type of elastomeric polymer matrix and fillers used.

Conductive elastomeric polymer may also be extruded and drawn into a filament in the presence of a gas to provide a desirable characteristics to the filament. Under certain conditions, oxygen may react with polymer materials to form crosslinked species (e.g. gels) which may have different properties from the bulk polymer. Non-uniform properties may result in broken filaments during spinning and drawing which may not be desirable. In an example, non-uniform properties may be mitigated against by sealing elastomeric material, conductive filler, and resulting filament in an inert environment (e.g. a nitrogen environment) sealed against air leaks may reduce the chance of the crosslinked species formation. In another example, inert gas (e.g. nitrogen) purging of an extruder feed throat may reduce crosslinked species formation.

Filaments from the melt spinning machine may be cooled to provide desirable characteristics. Filaments may be cooled in a quench system comprising convection heat transfer (e.g. to a gas) and/or conduction (e.g. to a liquid). Cooling gas flow may reduce along-a-filament denier and orientation variations in a filament. Applying different methods of cooling, including liquid bath (e.g. a water bath) during the melt spinning process may affect spun yarn birefringence (a measure of molecular orientation) which in turn correlates with spun yarn tenacity, % elongation, and initial modulus. Interactions between filament geometry and quench conditions may control spun fiber properties and their variability. In an example, a water bath may be used to cool extruded filaments from the melt spinning machine. In another example extruded filaments may be cooled by convection, e.g. by air convention. Filaments from the melt spinning machine may also be directed to a solvent bath, e.g. a water bath. The solvent bath may dissolve components of the filament to provide the remaining filament with a desirable shape or texture. The solvent bath may also cool the extruded filaments from the melt spinning machine. A cooling bath and/or solvent bath may be positioned a distance from a spinneret of the melt spinning machine to provide both cooling in the presence of a gas and a liquid to provide desirable characteristics of a filament.

Filaments according to the present disclosure may have different bi-component structures. Sometimes conductive pellets may not have sufficient mechanical strength to be extruded and drawn only by themselves; accordingly, another material may be used as a core material and the conductive material of the conductive pellets (which is made through compounding) may be a sheath. Filaments may have various structures such as hollow-fibers or a structures formed from polymer filaments extruded together in multi-component melt-spinning. The filaments may have various cross-section such as, for example, side-by-side, core and sheath, hollow, c-shape, trilobal, islands in the sea, and the like. In an example, an extruded filament may comprise water soluble polymer(s) (e.g. Poly(vinyl alcohol); “PVA”) which may be placed in a water bath after extrusion to remove the water soluble polymer(s). In another example, air may be blown during spinning to create hollow fibers where the sheath is formed from conductive polymer.

At block 1108, optionally filament yarn is formed from the filament(s). Yarn may be formed by a melt spinning machine illustrated at FIG. 15 illustration a). In an embodiment, after extruding the filament, the filament may be wrapped by water soluble polymer(s), e.g. PVA to make a yarn. In an example, mono-component filament comprising conductive elastomeric polymer, e.g. conductive TPE, may be a core wrapped by water soluble yarns (e.g. PVA) to make the yarn formed from the filament easier to knit. In another example, filament yarn described herein may be mono-filament or multi-filament yarn. In an embodiment, extruded filament may be coated with powder for better knittability. In an embodiment, the powder is Talc powder. As shown in FIG. 15 illustration b), the filament(s) or filament yarn may be further extruded to a desired dimension.

At block 1110, the yarn and/or filament is knit into an electrode. In an example, flat-bed knitting machines illustrated in FIG. 15 illustration c) may knit yarn or filament comprising conductive elastomeric polymer into electrodes (shown in FIG. 15 illustration d)) having a desired geometry and pattern. Based on the diameter of yarn the gauge of the knitting machine can be chosen. The thinner the yarn or filament, the higher the gauge of the machine to increase the resolution of the knitted electrode. In an example, different structures of electrode may be knit such as an electrode having a raised form factor or a flat form factor. The electrode may be made with various other textile manufacturing processes such as jacquard weaving, circular jacquard knitting, warp knitting and embroidery based on the desired properties of the electrode. For example jacquard weaving may be used to provide structures having improved dimensional stability; circular knitting may provide a structure with improved flexibility that may be produced quickly; and warp knitting may provide different yarn diagonals into knitted structures. Flat bed knitting may be used to apply different functionalities into the electrode, such as by inserting a functional laminate, RFID, and/or pH/sweat/moisture sensor behind the electrode during the manufacturing process. The size of the electrode, its geometry (e.g. square, oval, circular) may also be selected to improve performance for example by minimizing impedance at the skin-electrode interface. Yarn and/or filament may be knit into a desired pattern. Example patterns of knit filament are shown in FIG. 4 illustration c) which are enlarged views of a knit pattern under 20× and 100× magnification. The size, shape and materials of the yarn may be selected to enhance the performance of the electrode for receiving and/or recording a specific type of signal based on its frequency and amplitude range. In an example, an electrode comprises conductive thermoplastic elastomer, and the amount of conductive fillers, type of conductive filler, and structure of the yarn or filament such as diameter, elongation, tensile strength, cross-section, and geometrical structure may be varied to suit a desired application e.g. ECG, EMG, EEG, FES, etc.

An electrode according to the disclose herein may be used for different applications such that similar filament can be used in electrodes for bio-signal monitoring, functional electrical stimulation, heat generation, motion sensing, moisture sensing, respiration sensing, etc. Further, a single strand of the extruded filament may be knitted as an electrode such that material consumption may be reduced compared with other conductive filaments, e.g. carbon-contained nylon, silver plated nylon, etc. In some examples, because an extruded filament according to the disclosure herein may comprise silicone and/or rubber, an electrode made from the extruded element may have more grip when in contact with skin which may decrease motion artifact and retrieve bio-signals with higher resolution. In another example, electrodes according to the disclosure herein are biocompatible and such that they may be in contact with a human body for long-term monitoring and medical applications.

The electrode and/or conductive elastomeric filament fiber disclosed herein may also be used for strain gauge. In an embodiment, the resistance of a filament according to the disclose herein may change by stretching, causing the distance between conductive particles in filament matrix to change; in turn, causing resistance to change. By measuring the change in resistance as the electrode and/or filament may be used as a sensor for stretch/motion sensing.

The electrode and/or conductive elastomeric filament fibers disclosed herein may also be used for in heat applications. In an example, the conductive fillers, e.g. carbon-based fillers, may create high resistance so filament formed from conductive elastomeric materials, or a sheet of the conductive elastomeric materials, it can be used as a heating element by running an electric current through it. High conductivity yarns/filaments may be used as a bus and the extruded filament/sheet as heating element—due to the high resistance of sheet/filament, it will heat up and can be used in heat applications.

The electrode and conductive elastomeric filament fibers disclosed herein may also be used as a moisture sensor. The polymer matrix may be selected such that it's sensitive to a group of solvents and it swells once it comes in contact with those types of solvents/solutions therefore the distance between its conductive particles will change so its resistance will change and it can be sensitive to moisture.

The above description is meant to be exemplary only, and one skilled in the relevant arts will recognize that changes may be made to the embodiments described without departing from the scope of the invention disclosed. The present disclosure may be embodied in other specific forms without departing from the subject matter of the claims. The present disclosure is intended to cover and embrace all suitable changes in technology. Modifications which fall within the scope of the present invention will be apparent to those skilled in the art, in light of a review of this disclosure, and such modifications are intended to fall within the appended claims. Also, the scope of the claims should not be limited by the preferred embodiments set forth in the examples, but should be given the broadest interpretation consistent with the description as a whole.

Although the embodiments have been described in detail, it should be understood that various changes, substitutions and alterations can be made herein without departing from the scope. Moreover, the scope of the present disclosure is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As can be understood, the examples described above and illustrated are intended to be exemplary only.

### REFERENCES

[1] Y. J. Hong, H. Jeong, K. W. Cho, N. Lu, D. Kim, Advanced Functional Materials 2019, 29, 1808247.
[2] H. Carvalho, A. P. Catarino, A. Rocha, O. Postolache, In IEEE MeMeA 2014—IEEE International Symposium on Medical Measurements and Applications, Proceedings, IEEE Computer Society, 2014.
[3] C. O. Asogwa, V. Libeson, D. T. H. Lai, MEDICAL DEVICES & SENSORS 2018, 1, e10016.
[4] R. Paradiso, G. Loriga, N. Taccini, IEEE Transactions on Information Technology in Biomedicine 2005, 9, 337.
[5] G. J. Jin, M. J. Uddin, J. S. Shim, Advanced Functional Materials 2018, 28, 1804351.
[6] S. Myllymaa, A. Muraja-Murro, S. Westeren-Punnonen, T. Hukkanen, R. Lappalainen, E. Mervaala, J. Töyräs, K. Sipila, K. Myllymaa, Journal of Sleep Research 2016, 25, 636.
[7] R. Rajaganeshan, C. L. Ludlam, D. P. Francis, S. v. Parasramka, R. Sutton, International Journal of Clinical Practice 2007, 62, 65.
[8] Y. Khan, M. Garg, Q. Gui, M. Schadt, A. Gaikwad, D. Han, N. A. D. Yamamoto, P. Hart, R. Welte, W. Wilson, S. Czarnecki, M. Poliks, Z. Jin, K. Ghose, F. Egitto, J. Turner, A. C. Arias, Advanced Functional Materials 2016, 26, 8764.
[9] J. ANCZYKOWSKI, S. WILLEMS, B. A. HOFFMANN, T. MEINERTZ, S. BLANKENBERG, M. PATTEN, Journal of Cardiovascular Electrophysiology 2016, 27, 1032.
[10] D. Pani, A. Achilli, A. Bonfiglio, Advanced Materials Technologies 2018, 3, 1800008.
[11] D. Pani, A. Dessi, J. F. Saenz-Cogollo, G. Barabino, B. Fraboni, A. Bonfiglio, IEEE Transactions on Biomedical Engineering 2016, 63, 540.
[12] S. Yao, Y. Zhu, Nanomaterial-Enabled Dry Electrodes for Electrophysiological Sensing: A Review, Vol. 68, Minerals, Metals and Materials Society, 2016, pp. 1145-1155.
[13] A. Searle, L. Kirkup, Physiological Measurement 2000, 21, 271.
[14] P. J. Xu, H. Zhang, X. M. Tao, Textile Progress 2008, 40, 183.
[15] Y. Fu, J. Zhao, Y. Dong, X. Wang, Dry electrodes for human bioelectrical signal monitoring, Vol. 20, MDPI AG, 2020, pp. 1-30.
[16] C. Y. Huang, C. W. Chiu, ACS Applied Electronic Materials 2021.
[17] D. Hu, T. Cheng, K. Xie, R. Lam, Sensors 2015, 15, 26906.
[18] P. Ask, P. Å. Öderg, S. Ödman, T. Tenland, M. Skogh, Acta Anaesthesiologica Scandinavica 1979, 23, 189.
[19] L. Eskandarian, E. Lam, C. Rupnow, M. A. Meghrazi, H. E. Naguib, ACS Applied Electronic Materials 2020, 2, 1554.
[20] B. A. Reyes, H. F. Posada-Quintero, J. R. Bales, A. L. Clement, G. D. Pins, A. Swiston, J. Riistama, J. P. Florian, B. Shykoff, M. Qin, K. H. Chon, IEEE Transactions on Biomedical Engineering 2014, 61, 1863.
[21] Y. T. Tsukada, M. Tokita, H. Murata, Y. Hirasawa, K. Yodogawa, Y. ki Iwasaki, K. Asai, W. Shimizu, N. Kasai, H. Nakashima, S. Tsukada, Heart and Vessels 2019, 34, 1203.
[22] Y. Lin, M. Bariya, A. Javey, Wearable Biosensors for Body Computing, Wiley-VCH Verlag, 2020, p. 2008087.
[23] Performance Testing of Textiles, Elsevier, 2016.
[24] A. Miyamoto, S. Lee, N. F. Cooray, S. Lee, M. Mori, N. Matsuhisa, H. Jin, L. Yoda, T. Yokota, A. Itoh, M. Sekino, H. Kawasaki, T. Ebihara, M. Amagai, T. Someya, Nature Nanotechnology 2017, 12, 907.
[25] A. Soroudi, N. Hernandez, L. Berglin, V. Nierstrasz, Electrode placement in electrocardiography smart garments: A review, Vol. 57, Churchill Livingstone Inc., 2019, pp. 27-30.
[26] S. Kabiri Ameri, R. Ho, H. Jang, L. Tao, Y. Wang, L. Wang, D. M. Schnyer, D. Akinwande, N. Lu, ACS Nano 2017, 11, 7634.
[27] J. W. Lee, K. S. Yun, Polymers 2017, 9.
[28] Y. Wang, Y. Qiu, S. K. Ameri, H. Jang, Z. Dai, Y. A. Huang, N. Lu, npj Flexible Electronics 2018, 2, 6.
[29] T. Vuorinen, K. Noponen, A. Vehkaoja, T. Onnia, E. Laakso, S. Leppänen, K. Mansikkamäki, T. Seppänen, M. Mäntysalo, Advanced Materials Technologies 2019, 4, 1900246.
[30] H. Yang, S. Ji, I. Chaturvedi, H. Xia, T. Wang, G. Chen, L. Pan, C. Wan, D. Qi, Y. S. Ong, X. Chen, ACS Materials Letters 2020, 2, 478.
[31] Y. M. Chi, T. P. Jung, G. Cauwenberghs, IEEE Reviews in Biomedical Engineering 2010, 3, 106.
[32] Y. Ko, J. Oh, K. T. Park, S. Kim, W. Huh, B. J. Sung, J. A. Lim, S. S. Lee, H. Kim, ACS Applied Materials and Interfaces 2019, 11, 37043.
[33] X. Cheng, C. Bao, X. Wang, F. Zhang, W. Dong, Applied Physics A: Materials Science and Processing 2019, 125, 876.
[34] 35 Y. H. Chen, M. op de Beeck, L. Vanderheyden, E. Carrette, V. Mihajlović, K. Vanstreels, B. Grundlehner, S. Gadeyne, P. Boon, C. van Hoof, Sensors (Switzerland) 2014, 14, 23758.
[35] T. Kim, J. Park, J. Sohn, D. Cho, S. Jeon, ACS Nano 2016, 10, 4770.
[36] S. L. Colyer, P. M. McGuigan, Journal of Sports Science and Medicine 2018, 17, 101.
[37] M. Alizadeh Meghrazi, Y. Tian, A. Mahnam, P. Bhattachan, L. Eskandarian, S. Taghizadeh Kakhki, M. R. Popovic, M. Lankarany, BioMedical Engineering Online 2020, 19.
[38] A. Levitt, D. Hegh, P. Phillips, S. Uzun, M. Anayee, J. M. Razal, Y. Gogotsi, G. Dion, Materials Today 2020, 34, 17.
[39] J. Zhang, S. Seyedin, S. Qin, P. A. Lynch, Z. Wang, W. Yang, X. Wang, J. M. Razal, Journal of Materials Chemistry A 2019, 7, 6401.
[40] S. Seyedin, S. Moradi, C. Singh, J. M. Razal, Applied Materials Today 2018, 11, 255.
[41] S. Seyedin, S. Uzun, A. Levitt, B. Anasori, G. Dion, Y. Gogotsi, J. M. Razal, Advanced Functional Materials 2020, 30, 1910504.
[42] A. Mirabedini, J. Foroughi, G. G. Wallace, Developments in conducting polymer fibres: From established spinning methods toward advanced applications, Vol. 6, Royal Society of Chemistry, 2016, pp. 44687-44716.
[43] Z. He, G. Zhou, J. H. Byun, S. K. Lee, M. K. Urn, B. Park, T. Kim, S. B. Lee, T. W. Chou, Nanoscale 2019, 11, 5884.
[44] X. Li, H. Hu, T. Hua, B. Xu, S. Jiang, Nano Research 2018, 11, 5799.
[45] P. Costa, C. Silvia, J. C. Viana, S. Lanceros Mendez, Composites Part B: Engineering 2014, 57, 242.
[46] P. Huang, Z. Xia, S. Cui, Materials and Design 2018, 142, 11.
[47] T. Li, L. F. Ma, R. Y. Bao, G. Q. Qi, W. Yang, B. H. Xie, M. B. Yang, Journal of Materials Chemistry A 2015, 3, 5482.
[48] Duarte Rafael Bento de Matos, Conductive elastomeric materials for continuous electrocardiogram (ECG) monitoring, 2019.
[49] J. G. Drobny, Handbook of Thermoplastic Elastomers, 2007.
[50] A. Damley-Strnad, B. Morales, C. Ellis, L. R. Grace, Electrical and mechanical properties of a biocompatible carbon black reinforced thermoplastic elastomer for sensing applications, DEStech Publications Inc., 2017, pp. 1875-1889.
[51] Y. Noh, J. R. Bales, B. A. Reyes, J. Molignano, A. L. Clement, G. D. Pins, J. P. Florian, K. H. Chon, Annals of Biomedical Engineering 2016, 44, 2464.
[52] Environment and Climate Change Canada—Evaluating Existing Substances—Screening Assessment for the Challenge Carbon Black.
[53] S. Risse, B. Kussmaul, H. Kruger, G. Kofod, RSC Advances 2012, 2, 9029.
[54] J. Xu, K. M. Razeeb, S. Roy, Journal of Polymer Science Part B: Polymer Physics 2008, 46, 1845.
[55] R. Padmanabhan, K. Naskar, G. B. Nando, Materials Research Express 2015, 2, 105301.
[56] Y. Wu, J. Liu, X. Jiao, F. Cheng, G. Lai, X. Yang, ACS Omega 2020, 5, 6199.
[57] J. Wu, J. Dong, Y. Wang, B. K. Gond, Polymer Degradation and Stability 2017, 135, 43.
[58] M. Chipara, K. Lozano, A. Hernandez, M. Chipara, Polymer Degradation and Stability 2008, 93, 871.
[59] J. R. Potts, O. Shankar, S. Murali, L. Du, R. S. Ruoff, Composites Science and Technology 2013, 74, 166.
[60] J. R. Potts, O. Shankar, L. Du, R. S. Ruoff, Macromolecules 2012, 45, 6045.
[61] R. J. Young, P. A. Lovell, Introduction to Polymers, 2011.
[62] Y. Lin, S. Liu, S. Chen, Y. Wei, X. Dong, L. Liu, Journal of Materials Chemistry C 2016, 4, 6345.
[63] K. Cherenack, L. van Pieterson, Smart textiles: Challenges and opportunities, Vol. 112, American Institute of PhysicsAIP, 2012, p. 091301.
[64] M. Siyao, S. Liu, Z. Peihua, L. Hairu, Polymers 2020, 12, 1.
[65] M. S. Spach, R. C. Barr, J. W. Haystad, E. C. Long, Circulation 1966, 34, 649.

1. A biocompatible yarn comprising:
a conductive elastomeric filament, the conductive elastomeric filament comprising a elastomeric polymer and conductive filler.

2. The biocompatible yarn of claim 1, wherein the conductive elastomeric filament has a ΔR/R₀ of less than 2.3 for 100% strain, where ΔR is change in resistivity (Ohm·m), and R₀ is resistivity at 0% strain.

3. The biocompatible yarn of claim 1, wherein the conductive elastomeric filament has a Young's modulus in the range of 1-13 MPa.

4. The biocompatible yarn of claim 1, comprising 39%-70% carbon and 30-61% elastomer, wherein the elastomer comprises silicone.

5. (canceled)

6. The biocompatible yarn of claim 1 comprising at least one of carbon polyolefin (CPO); carbon styrene butadiene copolymer (CSBC); Carbon Silicone rubber (CSR1); and carbon silicone rubber (CSR2).

7. (canceled)

8. (canceled)

9. A conductive elastomeric filament comprising a elastomeric polymer and conductive filler.

10. The conductive elastomeric filament of claim 9, wherein the conductive elastomeric filament has a ΔR/R₀ of less than 2.3 for 100% strain, where ΔR is change in resistivity (Ohm·m), and R₀ is resistivity at 0% strain.

11. The conductive elastomeric filament of claim 9, wherein the conductive elastomeric filament has a Young's modulus in the range of 1-13 MPa.

12. The conductive elastomeric filament of claim 9 comprising 39%-70% carbon and 30-61% elastomer, wherein the elastomer comprises silicone.

13. (canceled)

14. The conductive elastomeric filament of claim 9 comprising at least one of carbon polyolefin (CPO); carbon styrene butadiene copolymer (CSBC); Carbon Silicone rubber (CSR1); and carbon silicone rubber (CSR2).

15. The conductive elastomeric filament of claim 9, wherein the filament has a generally uniform diameter along a length of the filament.

16. A wearable dry textile comprising the biocompatible yarn of claim 1.

17. An electrode comprising the biocompatible yarn of claim 1, the electrode configured for at least one of Electrocardiogram (ECG) measurement, electromyograms (EMG) measurement, electroencephalograms (EEG) measurement, Electrooculogram (EOG) measurement, Electrogastrogram (EGG) measurement, Functional Electrical Stimulation (FES), Transcranial Current Stimulation (TCS), High-Frequency Alternating Current Stimulation, Neuromuscular Electrical Stimulation (NMES), Transcutaneous Electrical Nerve Stimulation (TENS), Sensing pressure, Sensing strain, Heat generation, and/or creating a tactile sensation.

18. The electrode of claim 17 wherein the conductive elastomeric filament is knitted and/or woven into the yarn, and wherein the electrode is made from the yarn.

19. A method of manufacturing a conductive elastomeric filament for an electrode, the method comprising:
providing elastomeric polymer pellets having desired material properties;
combining the elastomeric polymer pellets and conductive filler together to form a conductive elastomer;
extruding and drawing the conductive elastomer into a filament.

20. The method of claim 19, comprising forming an electrode from the filament, the electrode configured for at least one of Electrocardiogram (ECG) measurement, electromyograms (EMG) measurement, electroencephalograms (EEG) measurement, Electrooculogram (EOG) measurement, Electrogastrogram (EGG) measurement, Functional Electrical Stimulation (FES), Transcranial Current Stimulation (TCS), High-Frequency Alternating Current Stimulation, Neuromuscular Electrical Stimulation (NMES), Transcutaneous Electrical Nerve Stimulation (TENS), Sensing pressure, Sensing strain, Heat generation, and/or creating a tactile sensation, wherein the elastomeric polymer and conductive filler comprise biocompatible material for forming a biocompatible yarn and/or filament.

21. (canceled)

22. The method of claim 19, wherein the elastomeric polymer is at least one of polyolefin, styrene butadiene copolymer, and silicone rubber; and wherein the conductive filler is carbon black.

23. The method of claim 19, wherein extruding and drawing the conductive elastomer into the filament comprises melt spinning the filament.

24. (canceled)

25. (canceled)

26. The method of claim 19, comprising extruding and drawing the filament into a solvent bath, wherein the solvent bath comprises water.

27. (canceled)

28. (canceled)

29. (canceled)

30. The method of claim 19, comprising knitting and/or weaving the filament into a yarn.
